(19) United States
(12) Patent Application Publication
(10) Pub. No.: US 2025/0250399 A1
(43) Pub. Date: Aug. 7, 2025

(54) IRRADIATED POLYALKYLENE CARBONATE COMPOSITIONS

(71) Applicant: Prevent-Plus LLC, St. Louis, MO (US)

(72) Inventors: David Gindelberger, Ladue, MO (US); Angela Sutter, St. Louis, MO (US); Julie Morris, Wildwood, MO (US); Brad Chartrand, Saratoga Springs, NY (US)

(21) Appl. No.: 19/027,007

(22) Filed: Jan. 17, 2025

Related U.S. Application Data
(63) Continuation of application No. PCT/US2023/070495, filed on Jul. 19, 2023.
(60) Provisional application No. 63/368,847, filed on Jul. 19, 2022.

(51) Int. Cl. C08J 3/28; B29C 71/02; C08G 64/32; C08G 64/40; C08J 3/09; C08J 7/12

(52) U.S. Cl. CPC C08J 3/28; C08G 64/32; C08G 64/40; C08J 3/093; C08J 7/08; C08J 7/123; C08J 2369/00

(57) ABSTRACT

Provided herein are methods of making polyalkylene carbonate compositions. The methods provided herein may comprise one or more of the following steps: (1) a polymerization step wherein an alkylene oxide is contacted with carbon dioxide, optionally in the presence of a catalyst, thereby forming a polyalkylene carbonate; (2) a dissolution step wherein the polyalkylene carbonate is dissolved in an organic solvent, thereby forming a polyalkylene carbonate solution; (3) a heat treatment step wherein a polyalkylene carbonate composition is heated, thereby forming a heat-treated poyalkylene carbonate composition; and (4) an irradiation step wherein a composition comprising polyalkylene carbonate is exposed to ionizing radiation, thereby providing an irradiated polyalkylene carbonate composition

### CROSS REFERENCE

This application is a continuation of International Application Serial No. PCT/US2023/070495, filed Jul. 19, 2023, which claims the benefit of priority from U.S. Provisional Patent Application 63/368,847 filed on Jul. 19, 2022, each of which is incorporated herein by reference in its entirety.

### BACKGROUND

In recent years, there has been increasing interest in developing improved composition and methods for treatment of wounds. Historically, the most common wound dressings have been gauze and tape. Gauze dressings, however, suffer from a number of significant disadvantages. Gauze may not completely protect the wound from water, bacteria, dirt, external oxygen, and other foreign matter. Additionally, gauze can stick to wound tissue, causing wound damage during dressing changes, increasing the probability of infection, and lengthening recovery time.

Multi-layered dressings are an improvement over traditional gauze dressings. However, these products also suffer from a number of significant disadvantages. Multi-layered dressings can be stiff and bulky, and are still susceptible to permitting bacteria and foreign material to enter wounds. Additionally, multi-layered dressings often have a layer for absorbing wound exudate, which needs to be changed during wound healing; the dressing, however, can stick to the wound, causing damage during dressing changes.

More recently, a new class of liquid polymer-based wound dressings, or “liquid bandages,” has been developed. Liquid bandages typically comprise a polymer component dissolved in an organic solvent. The solution is applied directly to the wound, the solvent is evaporated or precipitated, or the solution is coagulated in situ leaving a film covering over the wound.

Liquid compositions comprising polyalkylene carbonate polymers are particularly advantageous as wound dressings. An exemplary polyalkylene carbonate composition is DuraDerm®, which is a medical device indicated for providing a covering over minor wounds and scrapes that are clean and dry. DuraDerm® consists solely of organic polyethylene carbonate polymer (7.5%- 10% by weight) dissolved in methylene chloride organic solvent (90%- 92.5% by weight). The formulation eradicates any organisms (bacteria, fungi, viruses) it comes in contact with through lysing of the cell membranes thereof. This is a result of the methylene chloride's activity against an infinite number of organisms. In particular, testing has shown that methylene chloride functions to kill microbes (bacteria, fungi, and viruses) on contact, that is, the methylene chloride functions as a microbicidal agent. As will be appreciated based upon the following disclosure, methylene chloride functions in the manner described above while it is in liquid form and prior to evaporation thereof as the polymer film is formed. The general composition of DuraDerm® is disclosed in U.S. Pat. No. 6,909,027, entitled METHOD OF FORMING AN IN-SITU FILM DRESSING AND THE COMPOSITION OF THE FILM-FORMING MATERIAL; in U.S. Pat. No. 7,119,246, entitled METHOD OF TREATING ACNE; and in U.S. Publication No. 2020/0188426 A1, all of which are hereby incorporated herein by reference.

Despite the success of polyalkylene carbonate wound dressings, however, some challenges remain. Liquid polymer compositions can be highly viscous, and are therefore difficult to apply to some types of wounds. In addition, while existing polyalkylene carbonate wound dressings adhere readily to skin, improved adhesion would be beneficial for wounds in high-friction locations (e.g., joints) or for patients engaged in high-contact or athletic activities.

It is therefore desirable to develop new polyalkylene carbonate wound dressings, and methods of preparing and using such dressings, that exhibit a reduced viscosity, an increased adhesiveness, or a combination thereof.

### SUMMARY

Provided herein are methods of making polyalkylene carbonate compositions.

The methods provided herein may comprise one or more of the following steps: (1) a polymerization step wherein an alkylene oxide is contacted with carbon dioxide, optionally in the presence of a catalyst, thereby forming a polyalkylene carbonate; (2) a dissolution step wherein the polyalkylene carbonate is dissolved in an organic solvent, thereby forming a polyalkylene carbonate solution; (3) a heat treatment step wherein a composition comprising polyalkylene carbonate is heated, thereby producing a heat-treated polyalkylene carbonate composition; and (4) an irradiation step wherein a composition comprising polyalkylene carbonate is exposed to ionizing radiation, thereby providing an irradiated polyalkylene carbonate composition

For example, provided herein is a method of making an irradiated polyalkylene carbonate composition, the method comprising providing a base composition comprising a polyalkylene carbonate; and exposing the base composition to ionizing radiation, thereby producing an irradiated polyalkylene carbonate composition.

Also provided herein is a method of reducing the concentration of unincorporated ether functions present in a polyalkylene carbonate solution comprising a polyalkylene carbonate and an organic solvent, the method comprising a heat treatment step wherein a composition comprising polyalkylene carbonate is heated, thereby producing a heat-treated polyalkylene carbonate composition.

Also provided herein is a composition comprising irradiated polyalkylene carbonate, wherein the polyalkylene carbonate has been exposed to a radiation dose of at least about 5 kGy. Also provided herein are heat-treated polyalkylene carbonate compositions. Such polyalkylene carbonate compositions may be produced, for example, using methods as provided herein.

Also provided herein is a method of treating an animal, the method comprising applying a polyalkylene carbonate compositions as provided herein to the skin of an animal. For example, the polyalkylene carbonate composition may be applied as a wound dressing.

Other objects and features will be in part apparent and in part pointed out hereinafter.

### DETAILED DESCRIPTION

Provided herein are compositions comprising polyalkylene carbonate polymers, methods for preparing such compositions, and methods of using such compositions as wound dressings.

As used herein, the term “polyalkylene carbonate,” or equivalently “poly(alkylene carbonate),” refers to a polymer of the form:

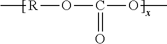

wherein R is a hydrocarbyl substituent and x is an average number greater than 1. For example, R may be a hydrocarbyl group having from 1 to about 10 carbon atoms, and more typically from about 2 to about 4 carbon atoms. Non-limiting examples of polyalkylene carbonates include polyethylene carbonate, polypropylene carbonate, and polybutylene carbonate.

The polyalkylene carbonate compositions provided herein may exhibit one or more improved properties that are advantageous in the context of a wound dressing. For example, the polyalkylene carbonate compositions provided herein may exhibit one or more of an improved viscosity; improved purity (e.g., reduced concentration of unincorporated ether functions, or UEFs); improved adhesiveness; and/or an increased breaking factor.

### Methods of Making

Methods of making the polyalkylene carbonate compositions provided herein may comprise one or more of the following steps: (1) a polymerization step wherein an alkylene oxide is contacted with carbon dioxide, optionally in the presence of a catalyst, thereby forming a polyalkylene carbonate; (2) a dissolution step wherein the polyalkylene carbonate is dissolved in an organic solvent, thereby forming a polyalkylene carbonate solution; (3) a heat treatment step wherein a polyalkylene carbonate composition is heated, thereby producing a heat-treated polyalkylene carbonate composition; and (4) an irradiation step wherein a composition comprising polyalkylene carbonate is exposed to ionizing radiation, thereby providing an irradiated polyalkylene carbonate composition. These steps are described in further detail below.

### Polymerization Step

The methods provided herein may comprise a polymerization step wherein an alkylene oxide is contacted with carbon dioxide, optionally in the presence of a catalyst, thereby forming a polyalkylene carbonate.

When the alkylene oxide is ethylene oxide, the polymerization step forms polyethylene carbonate as shown in the reaction scheme below.

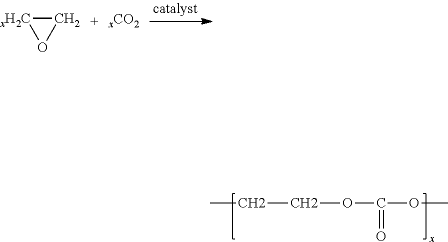

Preferably, the alkylene oxide comprises ethylene oxide, and the polyalkylene carbonate comprises ethylene carbonate. However, a variety of alkylene substituents can be employed in the polyalkylene carbonate polymer in order to achieve desired properties. Non-limiting examples of alkylene oxides that may be used include ethylene oxide, propylene oxide, butylene oxide, and mixtures thereof.

It is generally preferred to utilize polymers having lower alkylene substituents containing less than about 10 carbon atoms. For example, the polyalkylene carbonate may comprise an average of from about 2 to about 9 carbon atoms per constitutional repeating unit.

Suitable catalysts for the preparation of polyalkylene carbonates are known in the art. Non-limiting examples of suitable catalysts include zinc glutartate, zeolite-P₂S₅-metal catalysts, and Et₂Zn/ethylene glycol catalysts.

Polyalkylene carbonates prepared using a polymerization step as described above typically have a molecular weight of 50,000 Daltons or more. For example, polyethylene carbonates prepared using a polymerization step as described above typically have a molecular weight of from about 50,000 Daltons to about 200,000 Daltons.

### Dissolution Step

The methods provided herein may further comprise a dissolution step wherein the polyalkylene carbonate is dissolved in an organic solvent, thereby forming a polyalkylene carbonate solution.

The organic solvent is preferably a biocompatible organic solvent. Non-limiting examples of suitable solvents include methylene chloride, dichloroethane, propylene carbonate, dimethylformamide, N-Methyl pyrrolidone, acetone, ethyl acetate, tetrahydrofuran, methyl ethyl ketone as well as other ketones, esters, and ethers. A preferred solvent is methylene chloride.

The polyalkylene carbonate solution typically comprises the polyalkylene carbonate in an amount of from about 5% by weight to about 60% by weight, based upon the total weight of the solution. More typically, the polyalkylene carbonate solution comprises the polyalkylene carbonate in an amount of about 10% by weight to about 30% by weight, for example, about 15% by weight to about 25% by weight. For example, the polyalkylene carbonate solution may comprise the polyalkylene carbonate in an amount of at least about 2.5%, at least about 5%, at least about 10%, or at least about 15% by weight. The polyalkylene carbonate solution may comprise the polyalkylene carbonate in an amount of at most about 60%, at most about 50%, at most about 40%, at most about 30%, or at most about 25% by weight.

### Heat Treatment Step

The methods provided herein may further comprise a heat treatment step wherein a polyalkylene carbonate composition is heated, thereby forming a heat-treated polyalkylene carbonate composition.

Without being bound to a particular theory, when a polyalkylene carbonate is prepared using a polymerization step as described above, it is believed that at least two sources of impurities are typically present. First, some amount of alkylene oxide may fail to polymerize, and as a result, unreacted alkylene oxides may be present in the polyalkylene carbonate solution. Second, the alkylene oxide starting material may undergo self-polymerization during the polymerization step, thereby forming a polyalkylene oxide. As a result, the polyalkylene carbonate solution may further comprise polyalkylene oxides.

As used herein, the term “unincorporated ether functions, or “UEF,” refers collectively to any to alkylene oxides and/or polyalkylene oxides present in the polyalkylene carbonate solution. UEFs are generally undesirable. Without being bound to a particular theory, UEFs are believed to negatively impact the performance of the polyalkylene carbonate solution in at least three ways. First, an increased concentration of UEFs appears to correspondingly increase the viscosity of the solution, which will make a wound care product more difficult to apply. Second, an increased concentration of UEFs appears to correspondingly decrease the adhesion strength of a polymer film formed by the polyalkylene carbonate solution, which is obviously undesirable in the context of a wound care product. Third, high concentrations of UEFs have been observed to cause turbidity and haze in the polyalkylene carbonate solution, and can even lead to physical phase separation at sufficiently high concentrations.

Surprisingly, it has been discovered that heating a polyalkylene carbonate composition acts to release UEFs (e.g., free ethylene oxide, polyethylene oxide) entrapped in the composition.

The polyalkylene carbonate composition may be, for example, a polyalkylene carbonate solution formed by a dissolution step as described above. Alternatively, the polyalkylene carbonate composition may be an isolated polyalkylene carbonate polymer comprising one or more impurities. For example, the polyalkylene carbonate composition may be a solid precipitate formed by contacting a polyalkylene carbonate solution (e.g., a solution of polyalkylene carbonate in methylene chloride) with an anti-solvent such as methanol or acetone.

The heat treatment step may comprise heating a polyalkylene carbonate composition to a temperature of at least about 50° C., at least about 60° C., at least about 70° C., at least about 75° C., at least about 80° C., at least about 85° C., or at least about 90° C. For example, the heat treatment step may comprise heating the polyalkylene carbonate composition to a temperature of from about 70° C. to about 100° C., from about 75° C. to about 100° C., or from about 75° C. to about 90° C.

The heat treatment step may comprise heating the polyalkylene carbonate composition for a period of at least about 5 minutes, at least about 10 minutes, at least about 20 minutes, at least about 30 minutes, at least about 1 hour, at least about 1.5 hours, at least about 2 hours.

Following the heat treatment step, the polyalkylene carbonate composition preferably has a UEF concentration of no greater than about 5%, as compared to the overall abundance of carbonate groups in the composition. For example, the polyalkylene carbonate composition may have a UEF concentration of no greater than about 4%, no greater than about 3.5%, no greater than about 3%, no greater than about 2.5%, or no greater than about 2% as compared to the overall abundance of carbonate groups in the composition.

### Irradiation Step

The methods provided herein may further comprise an irradiation step wherein a composition comprising polyalkylene carbonate is exposed to ionizing radiation, thereby providing an irradiated polyalkylene carbonate composition.

It has been discovered that irradiation of polyalkylene carbonate will act to reduce its average molecular weight. It has been further discovered that irradiated polyalkylene carbonate compositions exhibit a number of surprising advantages. For example, irradiated polyalkylene carbonate compositions exhibit a lower viscosity than corresponding, non-irradiated compositions. Additionally, irradiated polyalkylene carbonate compositions exhibit better film adhesion than corresponding, non-irradiated compositions. These advantages are particularly significant and beneficial in the context of a wound dressing, which directly benefits from low viscosity (easier to apply) and increased adhesion (longer lasting).

The irradiation step may comprise exposing the polyalkylene carbonate to an electron beam, ultraviolet radiation, x-ray radiation, gamma radiation, or a combination thereof. For example, the polyalkylene carbonate may be exposed to radiation having an energy of at least about 10 eV, at least about 20 eV, at least about 40 eV, or at least about 50 eV. Preferably, the irradiation step comprises exposing the polyalkylene carbonate to gamma radiation.

The irradiation step may comprise exposing the polyalkylene carbonate to a radiation dose of at least about 5 kGy. Preferably, the radiation dose is at least about 20 kGy. For example, the irradiation step may comprise exposing the polyalkylene carbonate to a radiation dose of at least about 10 kGy, at least about 20 kGy, at least about 30 kGy, at least about 40 kGy, or at least about 50 kGy.

The irradiated polyalkylene carbonate composition preferably has an average molecular weight that is significantly lower than the starting material (i.e., the polyalkylene carbonate composition prior to the irradiation step). The irradiation step may comprise reducing the average molecular weight of the polyalkylene carbonate composition by at least about 10%, at least about 20%, at least about 30%, at least about 40%, at least about 50%, at least about 70%, or at least about 80% or more.

The irradiated polyalkylene carbonate composition may have an average molecular weight of less than about 150,000 Da, for example, less than about 125,000 Da, or less than about 100,000 Da. For example, the irradiated polyalkylene carbonate composition may have an average molecular weight of less than about 90,000 Da, less than about 80,000 Da, less than about 70,000 Da, less than about 60,000 Da, less than about 50,000 Da, or less than about 40,000 Da.

The irradiated polyalkylene carbonate composition preferably has a dynamic viscosity that is significantly lower than the starting material (i.e., the polyalkylene carbonate composition prior to the irradiation step). The irradiation step may comprise reducing the dynamic viscosity of the polyalkylene carbonate composition by at least about 10%, at least about 20%, at least about 30%, at least about 40%, at least about 50%, at least about 70%, or at least about 80% or more.

The irradiated polyalkylene carbonate composition may have a dynamic viscosity of less than about 100 cP, for example, less than about 90 cP, or less than about 80 cP. For example, the irradiated polyalkylene carbonate composition may have a dynamic viscosity of less than about 70 cP, less than about 60 cP, less than about 50 cP, less than about 40 cP, less than about 30 cP, less than about 20 cP, or less than about 10 cP.

### Polyalkylene Carbonate Composition

Also provided herein is a composition produced by a method as described above. For example, the composition may comprise a polyalkylene carbonate which has been irradiated and/or subjected to a heat treatment as described above.

### Compositions

In another aspect, the present disclosure is directed to compositions comprising polyalkylene carbonate (also referred to herein as “polyalkylene carbonate compositions”).

### Wound Dressing Composition

For example, provided herein is a polyalkylene carbonate composition that is useful as a wound dressing. The polyalkylene carbonate composition may be, for example, a polyethylene carbonate composition.

In accordance with one embodiment, the polyethylene carbonate composition is as follows: a base solution of polyethylene carbonate is dissolved in methylene chloride to provide a solution in which the polyethylene carbonate is present in a concentration of 7.5%-10% by weight based upon the solution. The polyethylene carbonate composition should be stored in a glass or Teflon lined container, as it is known that methylene chloride reacts with plastics.

Those skilled in the art will appreciate that the use of polyethylene carbonate as the polyalkylene carbonate polymer in the composition provided herein will results in a polyethylene carbonate composition having excellent oxygen barrier properties, a low glass transition temperature (Tg) of about 22° C., and very high elongation and recovery, flexibility and elasticity, which provides excellent conformity and protection to irregular body shapes. When used as a wound dressing, he low Tg exhibited by polyethylene carbonate permits body skin temperatures to soften the polymer further and better conform to irregular shapes, increasing the patient's comfort and providing excellent protection to the treatment area. Film thickness can be from about 0.25 mils to greater than about 3.0 mils, e.g., about 3.5 mils.

In certain cases, external oxygen may be desired and, therefore, polypropylene carbonate can be used, since it is not a good oxygen barrier. For example, and in accordance with another embodiment, a base solution of polypropylene carbonate having a glass transition temperature (Tg) of 40° C. is dissolved in methylene chloride to provide a solution in which the polyethylene carbonate is present in a concentration of 7.5%- 10% by weight based upon the solution.

By blending polyethylene carbonate polymers, e.g., polypropylene carbonate and polyethylene carbonate, either physically or chemically (terpolymer), intermediate properties can be obtained to optimize treatment.

### Molecular Weight

The polyalkylene carbonate composition may comprise a polyalkylene carbonate having an average molecular weight of less than about 150,000 Da, for example, less than about 125,000 Da, or less than about 100,000 Da. For example, the polyalkylene carbonate composition may have an average molecular weight of less than about 90,000 Da, less than about 80,000 Da, less than about 70,000 Da, less than about 60,000 Da, less than about 50,000 Da, or less than about 40,000 Da.

### Viscosity

The polyalkylene carbonate composition may have a dynamic viscosity of less than about 100 cP, for example, less than about 90 cP, or less than about 80 cP. For example, the irradiated polyalkylene carbonate composition may have a dynamic viscosity of less than about 70 cP, less than about 60 cP, less than about 50 cP, less than about 40 cP, less than about 30 cP, less than about 20 cP, or less than about 10 cP.

### Exposure to Ionizing Radiation

The polyalkylene carbonate composition may comprise a polyalkylene carbonate polymer that has been irradiated. For example, the composition may comprise a polyalkylene carbonate polymer that has been exposed to a radiation dose of at least about 5 kGy, at least about 10 kGy, at least about 20 kGy, at least about 30 kGy, at least about 40 kGy, or at least about 50 kGy. The composition may comprise a polyalkylene carbonate polymer that has been exposed to ultraviolet radiation, x-ray radiation, gamma radiation, or a combination thereof.

### Purity

The polyalkylene carbonate composition preferably has a UEF concentration of no greater than about 5%, as compared to the overall abundance of carbonate groups in the composition. For example, the polyalkylene carbonate solution may have a UEF concentration of no greater than about 4%, no greater than about 3.5%, no greater than about 3%, no greater than about 2.5%, or no greater than about 2% as compared to the overall abundance of carbonate groups in the composition.

### Breaking Factor

The polyalkylene carbonate composition may exhibit a breaking factor that is advantageous for use as a wound dressing. As used herein, the term “breaking factor” refers to the maximum load endured by a film sample prior to breaking, divided by the sample width. For example, the polyalkylene carbonate composition may exhibit a breaking factor of greater than 1, greater than about 1.5, or even greater than about 2.

### Wound Treatment System

Also provided herein is a wound treatment system useful for delivering a predetermined quantity of a polyalkylene carbonate composition to the surface of a wound.

For example, the wound treatment system may comprise a “crushable glass ampoule” delivery device, which comprises a predetermined amount of the polyalkylene carbonate composition. As an illustrative example, the delivery device may comprise one or more of (a) a flexible cannula; (b) an ampoule containing a predetermined amount of the polyalkylene carbonate composition; (c) a filter, preferably comprising a natural or synthetic fiber or textile (e.g., cotton). In a preferred embodiment, the delivery device comprises a glass ampoule located within a flexible plastic cannula, which further comprises a cotton filter located at the distal end of the cannula. When the user squeezes the cannula, the glass ampoule ruptures and releases the predetermiend amount of the polyalkylene carbonate composition within the cannula. The polyalkylene carbonate composition then passes through the cotton filter and exits the cannula, forming a layer over the surface of the wound to be treated. Fragments f the ampoule are retained within the cannula, as they cannot pass through the filter. This delivery system therefore allows a user to safely and efficiently apply a predetermined quantity of the alkylene carbonate composition to a wound in need of bandaging.

Also provided herein is a method of bandaging a wound, the method comprising applying a polyalkylene carbonate composition as provided herein to the surface of a wound. A predetermined quantity of the polyalkylene carbonate composition may be applied to the surface of the wound, for example, using a wound treatment system as provided herein.

### EXAMPLES

The following non-limiting examples are provided to further illustrate the present disclosure.

### Example 1

Samples of polyethylene carbonate, as a 10% solution in methylene chloride, were evaluated for the presence of unincorporated ether functionalities. Samples were obtained from two different manufacturing batches provided by the same manufacturer, and are identified in Table 1 below.

TABLE 1
Sample Composition and Visibility

| Sample | | Visible |
|---|---|---|
| # | Composition | Characteristics |
| Sample 1 | Polyethylene carbonate, as a 10% solution | Cloudy |
| | in methylene chloride | |
| Sample 2 | Polyethylene carbonate, as a 10% solution | Clear |
| | in methylene chloride | |

A turbidity analysis method using a UV-VIS Spectrophotometer to measure absorbance at 750 nm was developed to quantitate the relative turbidity of the two polymer solutions. See FIG. 3. The turbidity of Sample 1 measured 13.7 NTU, whereas the turbidity of Sample 2 measured 3.4 NTU. This was consistent with the visually cloudy appearance of Sample 1, as compared to the clear appearance of Sample 2.

The polyethylene carbonate samples were then subjected to a comparative GC headspace analysis (AP SOP 9.8, Limit of Free Ethylene Oxide test), along with an ethylene oxide standard. The analysis indicated that Sample 1 may contain significant amounts of entrapped ethylene oxide (RT ˜6.65 min), in excess of that observed for Sample 2. Visually, Sample 1 clarified considerably after being heated to 80° C. for 30 minutes during the headspace analysis. This further indicated free ethylene oxide or polyethylene oxide might be the cause of the relative cloudiness of the Sample 1 solution.

### Example 2

Using the same methods as described in Example 1 above, a number of additional samples were tested to determine their molecular weight, viscosity, and turbidity. Each sample contained a 10% w/w solution of polyethylene carbonate in methylene chloride; the samples were drawn from different manufacturing batches prepared by the same manufacturer. NMR data for select samples was also obtained. The results are presented in Table 2 below.

TABLE 2
Summary of Sample Data

| | | | | | | | | NMR data | | | |
|---|---|---|---|---|---|---|---|---|---|---|---|
| | | | | Turbidity (NTU) | | | | % ECU | | % IEF | |
| | | | | after | after | after | | ethylene | % EF | Incorporated | % UEF/EO/PEO |
| Sample | Molecular | Solution | Viscosity (cP) | 24 | 96 | 264 | after 5 | carbonate | Ether | Ether | Unincorporated |
| # | Wt. | Appearance | after 24 hrs | hrs | hrs | hrs | months | units | function | Function | Ether Funct |
| 1 | 188,000 | Hazy | 600 | 14 | 6 | 7 | 1 | | | | |
| | | | 445^{a} | | | | | | | | |
| | | | 314^{b} | | | | | | | | |
| 2 | 157,000 | Clear | 84 | 5 | 5 | 5 | 2 | | | | |
| 3 | 230,000 | Clear | 361 | 6 | 6 | 6 | | | | | |
| 4 | 238,000 | Clear | 440 | 7 | 7 | 7 | 7 | | | | |
| 5 | 169,000 | Clear | 116 | 6 | 6 | 6 | | 96.47 | 1.73 | 1.52 | 0.21 |
| 6 | 63,000 | Clear | 19 | 5 | 3 | 3 | | 94.88 | 2.32 | 2.09 | 0.23 |
| 7 | 142,000 | Clear | 38 | 6 | 5 | 5 | | 95.46 | 2.11 | 1.81 | 0.3 |
| 8 | 187,000 | Hazy | 368 | 15 | 6 | 2 | 1 | 83.87 | 10.95 | 5.18 | 5.77 |
| | | | 346^{c} | | | | | | | | |
| 9 | 178,000 | Slightly | 251 | 10 | 5 | 2 | 1 | 85.87 | 8.9 | 5.07 | 3.83 |
| | | Hazy | | | | | | | | | |
| 10 | 102,000 | Clear | 41 | 6 | 5 | 5 | | 87.9 | 7.23 | 4.92 | 2.31 |
| 11 | 173,000 | Clear | 200 | 3 | 3 | 3 | 1 | 88.24 | 7.02 | 4.82 | 2.2 |

^{a}After 10 days the solution had cleared significantly, and a thin layer had separated at the top. The lower layer was sampled for viscosity.
^{b}The lower layer was sampled again after another 10 days and analyzed for viscosity.
^{c}After 10 days the solution had cleared significantly, there was no formation of a separate layer. The material was sampled mid-bottle and analyzed for viscosity.

A general trend of increasing viscosity with increasing molecular weight was observed.

Samples with turbidity values at or below 7 NTU appeared visually clear. Samples with turbidity values around 10 NTU were slightly visually hazy, and samples with turbidity values of about 14 NTU or greater were noticeably hazy.

Without being bound to a particular theory, there was observed to be a correlation between the impurity and the viscosity of the samples. As the haziest samples clarified, the viscosity decreased and appeared to be more in line with the relationship between molecular weight and viscosity demonstrated by the other, clearer, polymer solutions.

The turbidity of all the solutions were monitored over a time frame of approximately 10 days. Interestingly, it was noted that the haziest solution (Sample 1) began to form a separate layer on top of the bulk of the solution after 10 days. The majority of the solution, the lower layer, began to clarify. This bottom layer was sampled periodically and reanalyzed for both viscosity and turbidity. This data is included in Table 2 above.

FTIR analysis of the top layer which formed in Sample 1A indicated the presence of polyethylene oxide. Specifically, the FTIR spectra indicated the presence of polyethylene carbonate in the top layer with maxima at 1740 and 1200 cm⁻¹ as well as the presence of a broad peak at 2880 cm⁻¹ which is similar to the peak seen for an IR of polyethylene oxide (˜100,000 Mwt) in the same region. This data provided further support supported the theory that free ethylene oxide, or polyethylene oxide, might be the cause of the haziness noted in several of the polymer solutions.

Again without being bound to a particular theory, the cloudiness of the haziest solutions tested (Samples 1, 8, and 9) appears to be due to higher amounts of Unincorporated Ether Functions, believed to be a combination of ethylene oxide and polyethylene oxide, as impurities in the polyethylene carbonate polymer. Although the solutions clarified over time, the cloudiness was observed to negatively impact the product in three significant ways: (1) The impurity appeared to increase the viscosity of the product solution which may make application of the product more difficult particularly when using the product in a pen applicator or spray package configuration. (2) The presence of the impurity in the solution appeared to decrease the adhesion strength of the polymer film formed. (3) The haze and potential for subsequent formation of a separate layer is undesirable from both a quality and aesthetic perspective.

### Example 3

In order to evaluate the characteristics of liquid bandage films formed from solutions formulated with polyethylene carbonate of various molecular weights, multiple film samples were formed on smooth aluminum sheets. Films formed from samples containing polyethylene carbonate of lower molecular weight were noticeably more difficult to remove from the aluminum surface.

Although an attempt was made to control the relative surface area and solution weight applied, it was difficult to make any quantitative comparisons regarding adhesion as the films did not dry to a uniform thickness. However, when testing the film samples once removed from the surface, the Breaking Factor (maximum load endured by a film sample prior to breaking divided by the sample width) indicated films formed using lower molecular weight PEC had lower breaking factors. These results are presented in Table 3 below.

TABLE 3
Film Tensile Testing Results

| | Molecular | Breaking |
|---|---|---|
| Sample # | Weight | Factor |
| Sample 6 | 65,000 | 1.08 |
| Sample 10 | 102,000 | 2.14 |
| Sample 2 | 157,000 | 2.25 |

### Example 4

The observation of increased adhesion of films formed from lower molecular weight polyethylene carbonate was also in line with observations made when using compositions packaged in single use applicator sticks (PREVENTOGEN Single Use Applicator Sticks) as compared before and after 25 kGy irradiation. Multiple users reported that the sticks “worked better” following irradiation. The formulation flowed more easily from the irradiated sticks (flow immediate following cracking and inversion for irradiated versus 15 seconds of squeezing required following cracking and inversion of non-irradiated) and the film formed seemed to adhere more securely. A sample solution (polyethylene carbonate, 10% w/w in methylene chloride) was prepared, and the molecular weight of the polyethylene carbonate in the solution was measured for a non-irradiated sample, a 20 kGy irradiated sample, and a 50 kGy irradiated sample. It was observed that the molecular weight of the polyethylene carbonate in solution was reduced following irradiation, as was the viscosity of the solution. These data are presented in Table 4 below.

TABLE 4
Impact of Irradiation on Polymer Molecular
Weight and Formulation Viscosity

| | Molecular | Viscosity |
|---|---|---|
| Sample # | Weight | (cP) |
| Sample 2 (control - no irradiation) | 133,260 | 80 |
| Sample 2 - 20 kGy | 63,160 | 16 |
| Sample 2 - 50 kGy | 39,865 | 9 |

Although the correlation of increased solution viscosity as the molecular weight of the polyethylene carbonate increases was anticipated, the observation of better film adhesion with lower molecular weight PEC was unexpected.

### Example 5

An in vivo study was conducted to observe differences in film adhesion on the skin. A test subject washed both arms with soap and water followed by thorough drying with a paper towel. Four sections were created on the underside of the right forearm, and three on the top of the left forearm using medical tape. Each formulation (listed in Table 5 below) was swabbed over the full area, allowed to dry, and the medical tape removed. The test subject was asked to submit pictures of the test areas after 24, 48, and 96 hours.

All four formulations adhered well to the skin. The location of the test swab appeared to be the strongest variable related to adhesion as the edge of the wrists on both arms and underside of the right underarm, which are locations most likely to experience friction during normal movement, were the first to wear off. The most noticeable difference related to molecular weight was the edges of the higher molecular weight formulations, DURADERM and Sample 11 were the more visible than those of the lower molecular weight formulations. This could have been due to removing the tape following drying rather than prior to drying.

TABLE 5
Results of Wear Study

| | Polymer | | |
|---|---|---|---|
| Sample | M_{w} | Location | Notes |
| Sample 6 | 65,000 | 3R, 3L | In place in both locations after 48 hours. |
| | | | In place on top of left arm after 96 hours. |
| Sample 10 | 102,000 | 1L | In place on top of left arm after 96 hours. |
| DURADERM | 133,000-157,000 | 1R, 2L | In place in both locations after 24 hours. |
| | | | In place on top of left arm after 96 hours. |
| Sample 11 | 173,000 | 2R, 4R | Location 4R showed significant wear after 24 |
| | | | hours, likely due to the location closer to the |
| | | | elbow which is more likely to be rubbed |
| | | | during normal movement. |
| | | | Location 2R remained in place after 48 hours. |

### Example 6

In order to determine the impact of irradiation and polyethylene carbonate molecular weight on the microbial barrier capacity of films formed using a polyethylene carbonate/methylene chloride solution, a test method utilizing differential, color changing agar was employed to indicate if the challenge organism had the capability to ingress through dried bandage into a media below. Multiple solutions formulated with various lots of polyethylene carbonate were evaluated both pre-and post-irradiation. The data collected using this study are summarized in Table 8 below.

TABLE 8
Microbial Barrier Challenge Test Results

| | Molecular | Viscosity | Barrier Test |
|---|---|---|---|
| Sample | Weight | (cP) | Results |
| Sample 10 | 102,000 | 52 | No growth all 4 |
| | | | organisms |
| Sample 10 - 50 kGy | 29,109 | 9 | No growth all 4 |
| | | | organisms |
| Sample 11 | 173,000 | 200 | No growth all 4 |
| | | | organisms |
| Sample 11 - 50 kGy | 32,712 | 8 | No growth all 4 |
| | | | organisms |
| Sample 4 | 238,000 | 440 | No growth all 4 |
| | | | organisms |

The barrier challenge results indicate that PEC forms an effective microbial barrier over a full range of PEC molecular weights (30,000-240,000 Daltons). Gamma irradiation of the polyethylene carbonate solution with doses up to 50 kGy had no negative impact on barrier performance.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

1. A method of making an irradiated polyalkylene carbonate composition, the method comprising:
(1) a polymerization step wherein an alkylene oxide is contacted with carbon dioxide, optionally in the presence of a catalyst, thereby forming a polyalkylene carbonate;
(2) a dissolution step wherein the polyalkylene carbonate is dissolved in an organic solvent, thereby forming a polyalkylene carbonate solution; and
(3) an irradiation step, wherein a composition comprising polyalkylene carbonate is exposed to ionizing radiation, thereby providing an irradiated polyalkylene carbonate composition.

2. A method of making an irradiated polyalkylene carbonate composition, the method comprising:
providing a base composition comprising a polyalkylene carbonate; and
exposing the base composition to ionizing radiation, thereby producing an irradiated polyalkylene carbonate composition.

3. The method of claim 2 wherein the average molecular weight of the polyethylene carbonate in the irradiated polyalkylene carbonate composition is reduced by at least about 20% as compared to the average molecular weight of the polyethylene carbonate in the base composition.

4. The method of claim 2 wherein the dynamic viscosity of the irradiated polyalkylene carbonate composition is at least about 10% lower than the dynamic viscosity of the base composition.

5. The method of claim 1 wherein the polyalkylene carbonate solution is exposed to ionizing radiation selected from the group consisting of an electron beam, ultraviolet radiation, x-ray radiation, gamma radiation, and combinations thereof.

6. The method of claim 5 wherein the polyalkylene carbonate solution is exposed to an electron beam.

7. (canceled)

8. The method of claim 1 wherein the polyalkylene carbonate solution is exposed to a radiation dose of at least about 5 kGy.

9. The method of claim 1 wherein the irradiated polyalkylene carbonate composition comprises polyalkylene carbonate having an average molecular weight of or less than about 100,000 Da.

10. (canceled)

11. (canceled)

12. A method of reducing the concentration of unincorporated ether functions present in a composition comprising a polyalkylene carbonate, the method comprising:
a heat treatment step wherein the composition is heated, thereby releasing unreacted alkylene oxide from the composition and producing a heat-treated polyalkylene carbonate composition.

13. (canceled)

14. (canceled)

15. (canceled)

16. (canceled)

17. (canceled)

18. The method of claim 1 wherein the polyalkylene carbonate is selected from the group consisting of polyethylene carbonate, polypropylene carbonate, and mixtures thereof.

19. The method of claim 1 wherein the organic solvent comprises methylene chloride.

20. The method of claim 1 wherein the polyalkylene carbonate solution typically comprises the polyalkylene carbonate in an amount of from about 5% by weight to about 60% by weight, based upon the total weight of the solution.

21. A polyalkylene carbonate composition prepared according to the method of claim 1.

22. (canceled)

23. (canceled)

24. The composition of claim 21 wherein the composition comprises polyalkylene carbonate having an average molecular weight of or less than about 100,000 Da.

25. (canceled)

26. The composition of claim 21 wherein the composition has a dynamic viscosity of less than about 70 cP.

27. The composition of claim 21 wherein the composition has a breaking factor of greater than 1.

28. The composition of claim 27 further comprising an organic solvent.

29. (canceled)

30. The composition of claim 28 comprising the polyalkylene carbonate in an amount of from about 5% by weight to about 60% by weight, based upon the total weight of the composition.

31. A method of treating an animal, the method comprising applying the composition of claim 21 to the skin of an animal.

32. A wound treatment system comprising:
(a) a flexible cannula;
(b) an ampoule containing a predetermined amount of the polyalkylene carbonate composition of claim 21, said ampoule being located within the flexible cannula; and
(c) a filter comprising a natural or synthetic fiber or textile, said filter being located within the flexible cannula.
